(19) United States
(12) Patent Application Publication
(10) Pub. No.: US 2009/0157145 A1
(43) Pub. Date: Jun. 18, 2009

(54) Transfer Coil Architecture

(76) Inventor: Lawrence Cauller, Plano, TX (US)

Correspondence Address: DAVID C. CAIN, PO Box 863387, Plano, TX, 75086-3387, US

(21) Appl. No.: 12/323,904

(22) Filed: Nov. 26, 2008

Related U.S. Application Data
(60) Provisional application No. 60/990,278, filed on Nov. 26, 2007.
(60) Provisional application No. 61/088,774, filed on Aug. 14, 2008.

(51) Int. Cl. A61N 1/08

(52) U.S. Cl. 607/60

(57) ABSTRACT

A system of wireless microtransponders, each including a RF resonator circuit for wireless power induction. An external power coil transmits RF energy at a matching or harmonic frequency to deliver power by near field induction to an intermediate, subcutaneous coil. Power is initially transmitted to a subdermal coil and relayed to the subcutaneous coil. The subcutaneous coil is used to transfer the RF signal and power the microtransponder using the resonator circuit. The external power coil RF frequency is tuned to match or be a harmonic of the micro-coil within the resonator.

### CROSS REFERENCE TO RELATED APPLICATIONS

This application claims priority from provisional patent application 60/990,278, filed on Nov. 26, 2007, and provisional patent application 61/088,774 filed on Aug. 14, 2008, which are hereby incorporated by reference.

### BACKGROUND

The numerous innovative teachings of the present application will be described with particular reference to a number of embodiments, including presently preferred embodiments (by way of example, and not of limitation), as well as other embodiments.

### BRIEF DESCRIPTION OF THE DRAWINGS

The disclosed inventions will be described with reference to the accompanying drawings, which show important sample embodiments of the invention and which are incorporated in the specification hereof by reference, wherein:

FIG. 1 is a functional schematic of a complete microtransponder for sensing and/or stimulating neural activity consistent with the present innovations.

FIG. 2 is an illustration of a laminar spiral micro-foil used in the construction of a microtransponder platform for stimulating neural activity consistent with the present innovations.

FIG. 3 is an illustration of a laminar spiral micro-coil electroplated onto a substrate consistent with the present innovations.

FIG. 4 is an illustration of a circuit diagram for a wireless microtransponder designed for independent auto-triggering operation (asynchronous stimulation) consistent with the present innovations.

FIG. 5 presents several graphs that summarize how wireless microtransponder stimulus frequency, stimulus current peak amplitude and stimulus pulse duration varies under different device settings and external RF power input conditions consistent with the present innovations.

FIG. 6 is an illustration of a circuit diagram for a wireless microtransponder with an external trigger signal de-modulator element to synchronize the stimuli delivered with a plurality other wireless microtransponders consistent with the present innovations.

FIG. 7 is a chart that illustrates de-modulation of an external interrupt trigger signal by differential filtering consistent with the present innovations.

FIG. 8 presents several graphs that summarizes the results from tests of a wireless microtransponder (with an external interrupt trigger de-modulator element) under different device settings and external RF power intensity conditions consistent with the present innovations.

FIG. 9A is an illustration of a deployment of a plurality of wireless microtransponders distributed throughout subcutaneous vascular beds and terminal nerve fields consistent with the present innovations.

FIG. 9B is an illustration of a deployment of wireless microtransponders to enable coupling with deep microtransponder implants consistent with the present innovations.

FIG. 9C is an illustration of a deployment of wireless microtransponders to enable coupling with deep neural microtransponder implants consistent with the present innovations.

FIG. 10 is an illustration of how wireless microtransponders can be deployed using a beveled rectangular hypodermic needle consistent with the present innovations.

FIG. 11 is an illustration of a fabrication sequence for spiral type wireless microtransponders consistent with the present innovations.

FIG. 12 is an illustration of an inner and outer transfer coil assembly using a coax cable.

### DETAILED DESCRIPTION OF THE PREFERRED EMBODIMENTS

A variety of medical conditions involve disorders of the neurological system within the human body. Such conditions may include paralysis due to spinal cord injury, cerebral palsy, polio, sensory loss, sleep apnea, acute pain, and so forth. One characterizing feature of these disorders may be, for example, the inability of the brain to neurologically communicate with neurological systems dispersed throughout the body. This may be due to physical disconnections within the neurological system of the body, and/or to chemical imbalances that can alter the ability of the neurological system to receive and transmit electrical signals, such as those propagating between neurons.

Advances in the medical field have produced techniques aimed at restoring or rehabilitating neurological deficiencies leading to some of the above-mentioned conditions. However, such techniques are typically aimed at treating the central nervous system and, therefore, are quite invasive. These techniques include, for example, implanting devices, such as electrodes, into the brain and physically connecting those devices via wires to external systems adapted to send and receive signals to and from the implanted devices. While beneficial, the incorporation of foreign matter into the human body usually presents various physiological complications, including surgical wounds and infection, which render these techniques potentially very challenging to implement with a risk of dangerous complications.

For example, the size of the implanted devices and wires extending therefrom may reduce or substantially restrict patient movement. Moreover, inevitable patient movements may cause the implanted device to shift, resulting in patient discomfort and possibly leading to the inoperability of the implanted device. Consequently, corrective invasive surgical procedures may be needed to reposition the device within the body, thereby further increasing the risk of infection and other complications.

In addition, an implanted device typically requires a battery to operate, and if the device is to remain within the body for prolonged periods, the batteries will need to be replaced, requiring additional surgical procedures that can lead to more complications. Furthermore, certain applications require that the implanted devices be miniaturized to the greatest extent possible, so they can be precisely implanted within the human body or so that a cluster of them can be implanted within a small defined area.

Publication US20020198572 by Weiner, for example, describes an apparatus for providing subcutaneous electrical stimulation. This device is certainly beneficial, providing pain relief by stimulating peripheral nerves, thus avoiding surgical interventions that target the brain or central nervous system (CNS). However, the device is bulky and has wire leads connecting the power sources to the implanted electrode.

Techniques such as those described in U.S. Publication 20030212440 by Boveja and related patents avoid the problem of battery replacement in a biostimulator by using a magnetic transmitter coil (RF transmission coil) placed over the region of the body that contains the implanted electrodes. This coil receives power and command signals via inductive coupling to generate stimulation pulses to activate motor units. Since the device contains no battery, the electrical power is derived from the externally generated RF field in the transmitting coil. However, this device is specifically designed for stimulus of the vagus nerve, and is not generally applicable to the current innovations. Further, the disclosed device still possesses a significant implant component with leads connecting the electrodes (alongside the vagus nerve) to the implanted stimulus receiver (in the chest).

Another approach is followed in devices similar to those described in U.S. Publication 20030212440 by Boveja made under the trademark BION® and currently in clinical trials for the treatment of urinary urge incontinence and headaches. The BION® units are fairly large, ranging about 2 mm×10 mm×2 mm (thickness), and much smaller embodiments are preferred for implantation. Furthermore, BION® units must be hermetically sealed in order to protect the coils from the damaging effects of water and other bodily fluids. Additionally, BION® units require relatively high levels of externally applied RF power (often >1 watt) to provide the greater stimulus currents necessary for their primary purpose to actively stimulate individual muscles or muscle groups.

U.S. Publication 20050137652 by Cauller et al. provides for small, wireless neural stimulators. In this disclosed device, a plurality of single channel electrodes interface with the cellular matter, thus allowing smaller devices to be used without sacrificing efficacy. Because the subdermal tissue conducts electrical signals, the small electrodes are able to provide sufficient signal for stimulating neurons, in spite of the devices small size and distance from the nerve.

U.S. Publication 20060206162 by Wahlstrand et al. also describes a device capable of transcutaneous stimulations with an array of electrodes that are attached to the skin surface on the back of the neck. However, this device contains a battery within the housing and is still quite large.

VeriChip® is the first FDA-cleared human-implantable RFID microchip. About twice the length of a grain of rice the device is glass-encapsulated (to seal the internal components away from the body), and implanted above the triceps area of an individual's right arm. Once scanned at the proper frequency, the VeriChip® responds with a unique sixteen-digit number which can correlate the user to information stored on a database for identity verification, medical records access and other uses. The data is not encrypted, causing serious privacy concerns, and there is some evidence that the devices may cause cancer in mice.

There are advantages to using even smaller, reliable, wireless implantable devices and/or methods adapted to treat neural or other biological disorders and to address aforementioned shortcomings.

The present application discloses new approaches to methods and apparatuses for providing minimally invasive wireless microtransponders that can be subdermaly implanted and configured to sense a host of biological signals and/or stimulate a variety of tissue responses. The microtransponders contain miniaturized micro-coils and a simplified circuit design to minimize the overall size of the microtransponders.

Power can be delivered externally using near field coupling to deliver power to subcutaneous implanted microtransponders. An external coil is used to deliver power to a subdermal coil via near field induction. The subdermal coil can be coupled by a tunable resonator circuit to a subcutaneous deep coil to deliver power to one or more proximate microtransponders by near field induction. Thus, four coils can be used to deliver power to the microtransponders (e.g., an external coil, a subdermal (or outer transfer) coil, a subcutaneous (or inner transfer) coil, and a microtransponder micro-coil).

The disclosed innovations, in various embodiments, provide one or more of at least the following advantages:
Remote RF power source option, eliminating requirement for bulky batteries and permitting deep stimulation.
The size and power advantages permit relatively complex digital electronics to be added to the smallest transponder.
Flexible deployment options allowing implantation at any depth and different powering options.
Allows microtransponder implantation at any point in the body.

The numerous innovative teachings of the present application will be described with particular reference to the presently preferred embodiment (by way of example, and not of limitation).

Various embodiments of the present invention are directed towards the miniaturization of minimally invasive wireless micro-implants termed “microtransponders,” which may be small enough to allow numerous independent microtransponders to be implanted under a square inch of skin for sensing a host of biological signals or stimulating a variety of tissue responses. The microtransponders can operate without implanted batteries or wires by receiving electromagnetic power from pliable coils placed on the surface of the overlying skin. The microtransponder design is based upon wireless technology Radio Frequency Identification Devices (RFIDs).

The present application discloses new approaches to methods and apparatuses for providing minimally invasive wireless microtransponders that can be subcutaneously implanted and configured to sense a host of biological signals and/or stimulate a variety of tissue responses. The microtransponders contain miniaturized micro-coils that are formed by utilizing novel fabrication methods and have simplified circuit designs that minimize the overall size of the microtransponders. The unprecedented miniaturization of minimally invasive biomedical implants made possible with this wireless microtransponder technology would enable novel forms of distributed stimulation or high resolution sensing using micro-implants so small that implantation densities of 100 per square inch of skin are feasible.

The simplicity of the microtransponders allows extreme miniaturization, permitting many microtransponders to be implanted into a given area, usually by relatively noninvasive injection techniques. The microtransponders are biologically compatible, thus avoiding the need to seal the devices (as with the VeriChip®) and further contributing to small size. Many biologically compatible materials and coatings are known, such as gold, platinum, SU-8, Teflon®, polyglycerols, or hydrophilic polymers such as polyethylene glycol (PEG). Additionally, many materials can be made biologically compatible by passivating the surface to render it non-reactive. In some embodiments, the microtransponder may include an anti-migration coating, such as a porous polypropylene polymer, to prevent migration away from the implant site. However, experiments to date indicate that the uncoated devices do not migrate. The tiny devices float independently in the tissue, moving only as the tissue moves, thus minimizing tissue rejection and encapsulation and maximizing longevity and effectiveness.

Wireless RFID technology involves the near-field magnetic coupling between two simple coils tuned to resonate at the same frequency (or having a harmonic that matches a harmonic or the fundamental frequency of the other coil). Throughout this document, references to tuning two coils to the “same frequency” includes having the frequencies of coils match at fundamental and/or harmonic frequencies. Radio Frequency (RF) electromagnetic power applied to one of these coils generates a field in the space around that power coil. Electrical power can be induced remotely in any remote coil placed within that power field as long as the remote coil is properly tuned to resonate at the same frequency as the power coil. However, tuning is not as critical at the inner boundary for inductive coupling.

An auto-triggering wireless microtransponder can be used to provide asynchronous electro-stimulation. The microtransponder of this embodiment includes a resonator element, a rectifier element, a stimulus voltage element, a stimulus discharger element, and a conducting electrode. The microtransponder is configured to discharge an electrical stimulus with a repetition rate that is controlled by the intensity of the externally applied RF power field.

A wireless microtransponder with an external trigger signal de-modulator element can be used to provide synchronized electro-stimulation. The microtransponder of this embodiment includes a resonator element, a rectifier element, an external trigger demodulator element, a stimulus timer element, a stimulus driver element, and a conducting electrode. The external trigger demodulator element is configured to receive a trigger signal from an external radio frequency (RF) power field. The stimulus driver element is configured to discharge an electrical stimulus when the external trigger demodulator element receives the trigger signal.

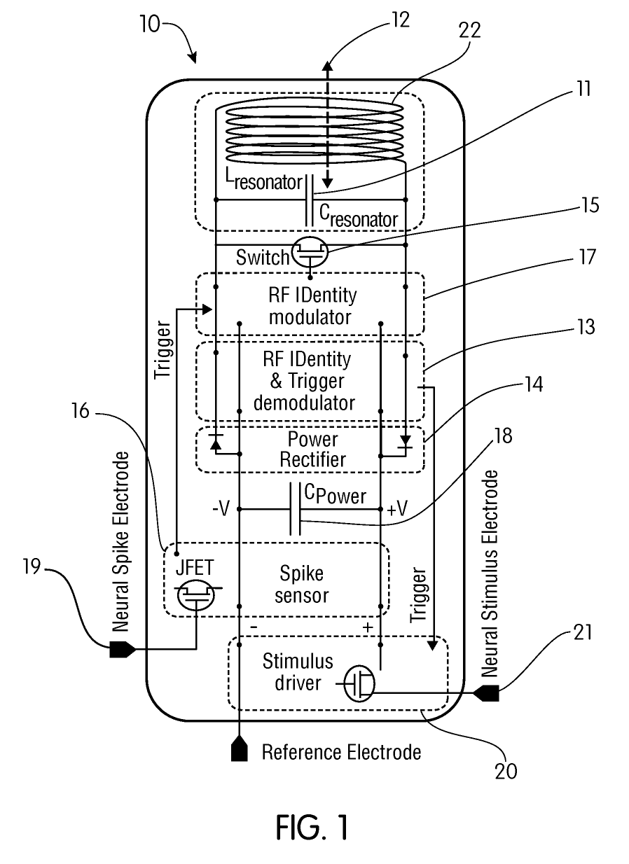

FIG. 1 is a functional schematic of a complete microtransponder for sensing and/or stimulating neural activity, in accordance with one embodiment. The circuit is designed for dependent triggering operation (synchronous stimulation). The circuit 10 includes electrical components adapted to electrically interface with neurons of peripheral nerves. The circuit 10 further includes electrical components which enable the microtransponder to wirelessly interact with systems external to the microtransponder. Such systems may include other transponders implanted within the body or external coils and/or a receiver. The wireless capabilities of the circuit 10 enable the delivery of electrical signals to and/or from the peripheral nerves. These include electrical signals indicative of neural spike signals and/or signals configured to stimulate peripheral nerves distributed throughout the subcutaneous tissue.

Accordingly, the circuit 10 includes the micro-coil 22 coiled about a central axis 12. The micro-coil 22 is coupled in parallel to a capacitor 11 and to an RF identity modulator 17 via a switch 15. The RF identity modulator 17 is coupled to an RF identity and trigger demodulator 13, which in turn is coupled to a rectifier 14. The rectifier 14 is coupled to a spike sensor trigger 16 and to a stimulus driver 20. The rectifier 14 and the spike sensor 16 are both coupled in parallel to a capacitor 18. In addition, the spike sensor 16 is coupled to a neural spike electrode 19, thereby electrically connecting the spike sensor 16 to neural transmission tissue (neurons). Similarly, the neural stimulus electrode 21 also connects the stimulus driver 20 to neural conduction tissue (axons). The spike sensor 16 is made up of one or more junction field effect transistors (JFET). As will be appreciated by those of ordinary skilled in the art, the JFET may include metal oxide semiconductors field effect transistors (MOSFETS).

The sensors, drivers, and other electronic components described in the present application maybe fabricated using standard small scale or very large scale integration (VLSI) methods. Further, the spike sensor 16 is coupled to the RF identity modulator 17, which is adapted to modulate an incoming/carrier RF signal in response to neural spike signals detected by the spike sensor 16. In one embodiment, the neural electrodes (i.e., neural spike electrode 19 and neural stimulus electrode 21) to which the spike sensor 16 and the stimulus driver 20 are connected, respectively, may be bundled and configured to interface with the neural conduction (axon) portion of a peripheral nerve.

One configuration of the above components, as depicted by FIG. 1, enables the microtransponder to operate as an autonomous wireless unit, capable of detecting spike signals generated by peripheral nerves, and relaying such signals to external receivers for further processing. It should be understood that the microtransponder performs such operations while being powered by external RF electromagnetic signals. The above-mentioned capabilities are facilitated by the fact that magnetic fields are not readily attenuated by human tissue. This enables the RF electromagnetic signals to sufficiently penetrate the human body so that signals can be received and/or transmitted by the microtransponder. In other words, the micro-coil 22 is designed and configured to magnetically interact with the RF field whose magnetic flux fluctuates within the space encompassed by the micro-coil 22. By virtue of being inductors, the micro-coils 22 convert the fluctuations of the magnetic flux of the external RF field into alternating electrical currents, flowing within the micro-coil 22 and the circuit 10. The alternating current is routed, for example, into the rectifier 14, which converts the alternating current into direct current. The direct current may then be used to charge the capacitor 18, thereby creating a potential difference across the JFET of the spike sensor 16.

In an exemplary embodiment, a gate of the spike sensor 16 JFET may be coupled via the neural spike electrode 19 to the neural transmission tissue (neurons). The gate of the spike sensor 16 JFET may be chosen to have a threshold voltage that is within a voltage range of those signals produced by the neural axons. In this manner, during spike phases of the neural axons, the gate of the spike sensor 16 becomes open, thereby closing the circuit 10. Once the circuit 10 closes, the external RF electromagnetic field generates an LC response in the coupled inductor 22 and capacitor 18, which then resonate with the external RF electromagnetic field, with its resonance matching the modulating frequency of the RF electromagnetic field. The LC characteristic of the circuit 10, as well as the threshold voltage of the gate of spike sensor 16 JFET, can be chosen to determine a unique modulation within the coupled micro-coil (i.e. inductor) 22 and capacitor 18, thereby providing an identifying signal for the microtransponder. Accordingly, the spike sensor 16 JFET provides the RF identity modulator 17 with a unique trigger signal for generating desired RF signals. The identity signal may indicate the nature of the neural activity in the vicinity of the microtransponder, as well as the location of the neural activity within the body as derived from the specific identified microtransponder position.

It should be appreciated that the RF capabilities, as discussed above with respect to the circuit 10, can render the microtransponder a passive device which reacts to incoming carrier RF signals. That is, the circuit 10 does not actively emit any signals, but rather reflects and/or scatters the electromagnetic signals of the carrier RF wave to provide signals having specific modulation. In so doing, the circuit 10 draws power from a carrier radio frequency (RF) wave to power the electrical components forming the circuit 10.

While the above-mentioned components illustrated in FIG. 1 may be used to receive signals from the microtransponder in response to spike signals generated by peripheral nerves, other components of circuit 10 of the microtransponder may include components for stimulating the peripheral nerves using the external RF signals. For example, the RF signals received by the micro-coil 22 may be converted to electrical signals, via the RF identity and trigger demodulator 13, so as to provide sufficient current and voltage for stimulating the peripheral nerves. Hence, the RF identity and trigger demodulator 13 derives power from an RF carrier signal for powering the stimulus driver 20, which delivers electrical signals suitable for stimulating neural conduction tissue (axons). This may be used to treat nerves that are damaged or that are otherwise physiologically deficient. Because of the nature of the identifying signal, a microtransponder can be selectively activated to provide electrostimulation.

It should be understood that, in certain embodiments, the minimum size for the microtransponders may be limited by the size of the micro-coil responsible for power induction, and secondarily by the size of the capacitors necessary for tuning power storage and timing. It should be understood that, in certain embodiments, the minimum size for the microtransponders may be limited by the size of the micro-coil responsible for power induction, and secondarily by the size of the capacitors necessary for tuning power storage and timing. In fact, micro-coils less than 1 millimeter in diameter and just a few micrometers thick can provide sufficient wireless power to operate the complex micro-electronics that can be manufactured on integrated circuit chips that may be much smaller than these coils. Combining the sophisticated functionality of micro-electronic chips with the wireless performance of these micro-coils creates the smallest possible, minimally invasive implants, in the form of tiny flecks as small as ˜0.1 mm thick and ˜1 mm wide. The size and power advantages make it possible to add relatively complex digital electronics to the smallest transponder.

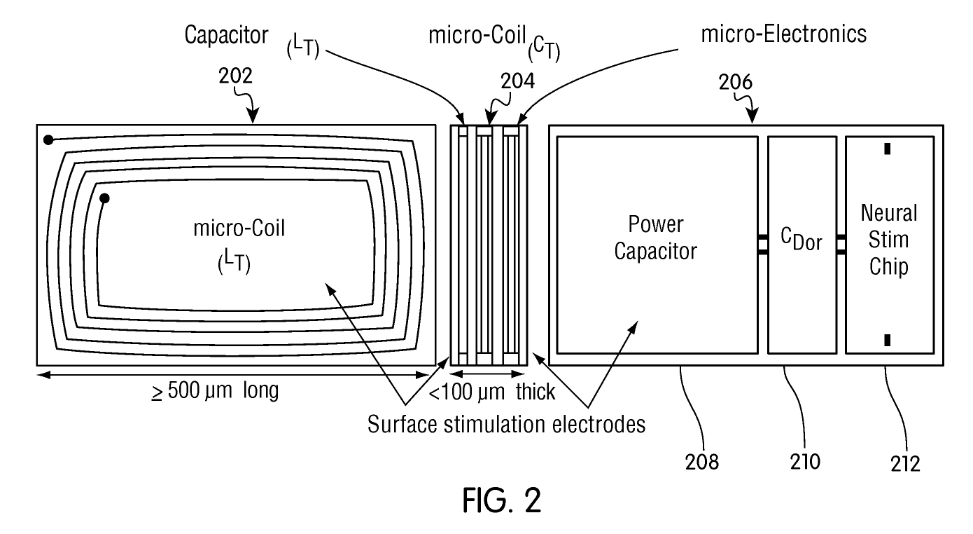

FIG. 2 is an illustration of a laminar spiral micro-coil power circuit used in the construction of a microtransponder platform for stimulating neural activity, in accordance with one embodiment. As depicted, herein, the microtransponder includes a laminar spiral micro-coil (L_{T}) 202 coupled to a capacitor (C_{T}) 204, which in turn is coupled to a microelectronics chip 206. The microelectronics chip 206 includes a power capacitor element 208 coupled to a capacitor (C_{DUR}) element 210, which in turn is coupled to a neural stimulation chip element 212. In an exemplary embodiment of the microtransponder platform, the micro-coil is no more than 500 μm long by 500 μm wide and the combined thickness of the laminar spiral micro-coil (L_{T}) 202, capacitor (C_{T}) 204, and micro-electronics chip 206 is no more than 100 μm.

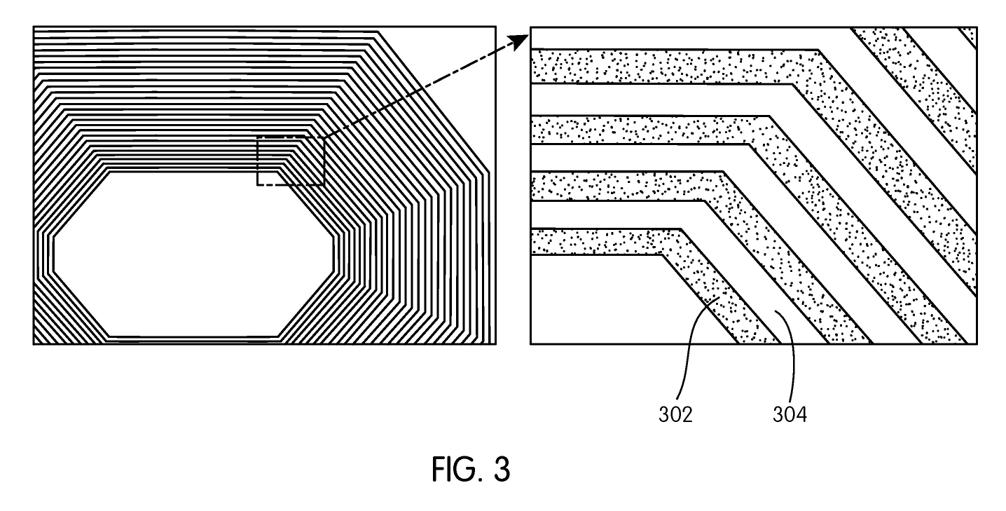

FIG. 3 is an illustration of a laminar spiral micro-coil electroplated onto a substrate, in accordance with one embodiment. As depicted in the drawing, conductor lines 302 are initially electroplated in a tight spiral pattern onto a non-reactive substrate (e.g., glass, silicon, etc.). In one embodiment, the laminar spiral micro-coil can include conductor lines 302 that are about 10 μm wide and the spacing 304 between the conductor lines 302 set at about 10 μm. In another embodiment, the laminar spiral micro-coil can include conductor lines 302 that are about 20 μm wide and the spacing 304 between the conductor lines 302 set at about 20 μm. It should be understood, however, that the widths of the conductor line 302 and line spacing 304 can be set to any value as long as the resulting micro-coil can produce the desired induced current for the desired application.

Platinum-iridium alloy is the preferred electroplating material to form the conductor lines 302. Gold or platinum are other acceptable conductors that can be utilized to form the conductor lines 302.

In certain embodiments, once the spiral micro-coil has been electroplated onto the substrate, a polymer-based layer is spun on top of the micro-coils to provide a layer of protection against corrosion and decay once implanted. Long-term studies of animals with SU-8 implants have verified the biocompatibility of SU-8 plastic by demonstrating that these SU-8 implants remain functional without signs of tissue reaction or material degradation for the duration of the studies. Therefore, typically, the polymer-based layer is comprised of an SU-8 or equivalent type of plastic having a thickness of approximately 30 μm.

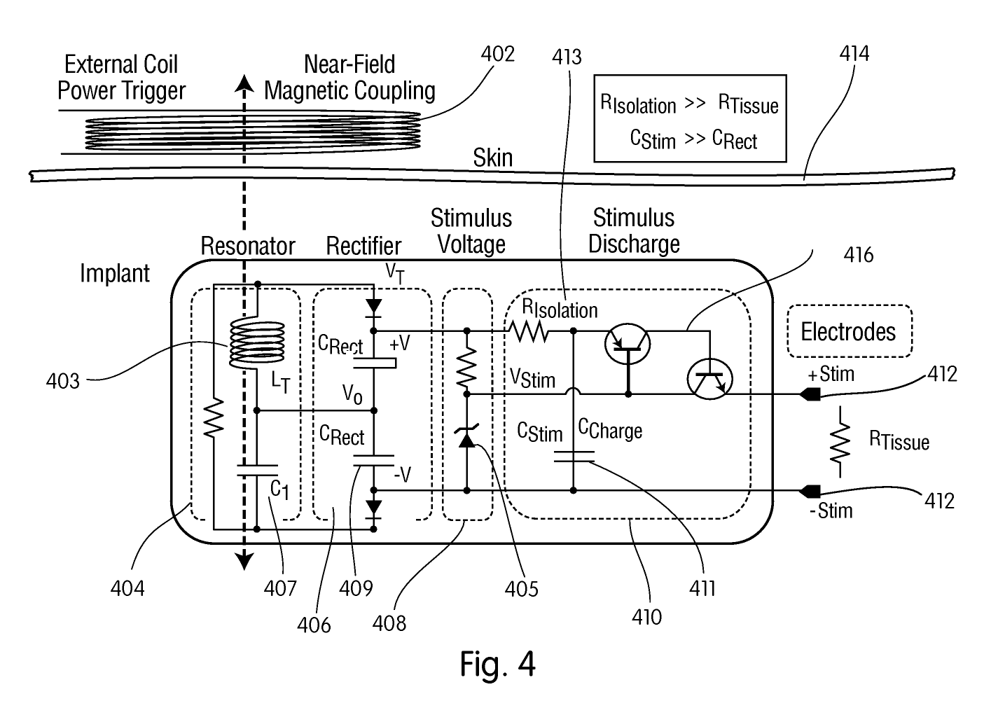

FIG. 4 is an illustration of a circuit diagram for a wireless microtransponder designed for independent auto-triggering operation (asynchronous stimulation), in accordance with one embodiment. As shown by the circuit diagram, the auto-triggering microtransponder includes a resonator element 404 (i.e., “tank circuit”), a rectifier element 406, a stimulus voltage element 408, a stimulus discharger element 410, and one or more electrodes 412. The resonator element 404 includes a coil (L_{T}) component 403 that is coupled to a capacitor (C_{T}) component 407. The resonator element 404 is configured to oscillate at a precise frequency that depends upon the values of these two components (i.e., the coil component 403 and capacitor component 407) as described in Equation 1:

Fᵣₑₛ=1/(2π√LC)

The resonator element 404 is coupled to the rectifier element 406, which is in turn coupled to the stimulus voltage element 408 and the stimulus discharger element 410. The rectifier element 406 and the stimulus voltage element 408 are both coupled in parallel to a capacitor 411. In addition, the stimulus discharger element 410 is coupled to electrodes 412, thereby electrically connecting the stimulus discharger element 410 to neural conduction tissue (axons). It should be appreciated that in certain embodiments, a voltage booster component (not shown) can be inserted immediately after the rectifier element 406 to boost the supply voltage available for stimulation and operation of integrated electronics beyond the limits generated by the miniaturized LC resonant ‘tank’ circuit 404. This voltage booster can enable electro-stimulation and other microtransponder operations using the smallest possible LC components which may generate too little voltage (<0.5V). Examples of high efficiency voltage boosters include charge pumps and switching boosters using low-threshold Schottky diodes. However, it should be understood that any type of conventional high efficiency voltage booster may be utilized in this capacity as long as it can generate the voltage required by the particular application of the microtransponder.

In this circuit configuration, the auto-triggering microtransponder can employ a bi-stable silicon switch 416 to oscillate between the charging phase that builds up a charge on the stimulus capacitor 411, and the discharge phase that can be triggered when the charge reaches the desired stimulation voltage by closing the switch 416 state to discharge the capacitor 411 through the stimulus electrodes 412. A single resistor 413 is used to regulate the stimulus frequency by limiting the charging rate. The breakdown voltage of a single zener diode 405 is configured to set the desired stimulus voltage by dumping current and triggering the switch 416 closure, discharging the capacitor 411 into the electrodes 412 (gold or Platinum-iridium alloy) when it reaches the stimulation voltage. Although gold was initially regarded as the preferred electrode material, it was discovered that in long-term implantation gold salt deposits could form and create a micro-battery, interfering with the stimulus signal. Gold remains a viable electrode material for some applications, but Platinum-iridium alloy is regarded as the preferred embodiment for long-term, permanent applications. Platinum is another acceptable electrode material.

The stimulus peak amplitude and duration are largely determined by the effective tissue (e.g., skin 414, muscle, fat etc.) resistance, independent of the applied RF power intensity. However, increasing the RF power may increase the stimulation frequency by reducing the time it takes to charge up to the stimulus voltage.

The auto-triggering microtransponder operates without timing signals from the RF power source (RF power coil) 402 and “auto-triggers” repetitive stimulation independently. As a result, the stimulation generated by a plurality of such auto-triggering microtransponders would be asynchronous in phase and somewhat variable in frequency from one stimulator to another depending upon the effective transponder voltage induced by each resonator circuit 404. While unique to this technology, there is no reason to predict that distributed asynchronous stimulation would be less effective than synchronous stimulation. In fact, such asynchronous stimulation may be more likely to evoke the sort of disordered “pins and needles” or “tingling” sensations of parasthesias that are associated with stimulation methods that most effectively block pain signals.

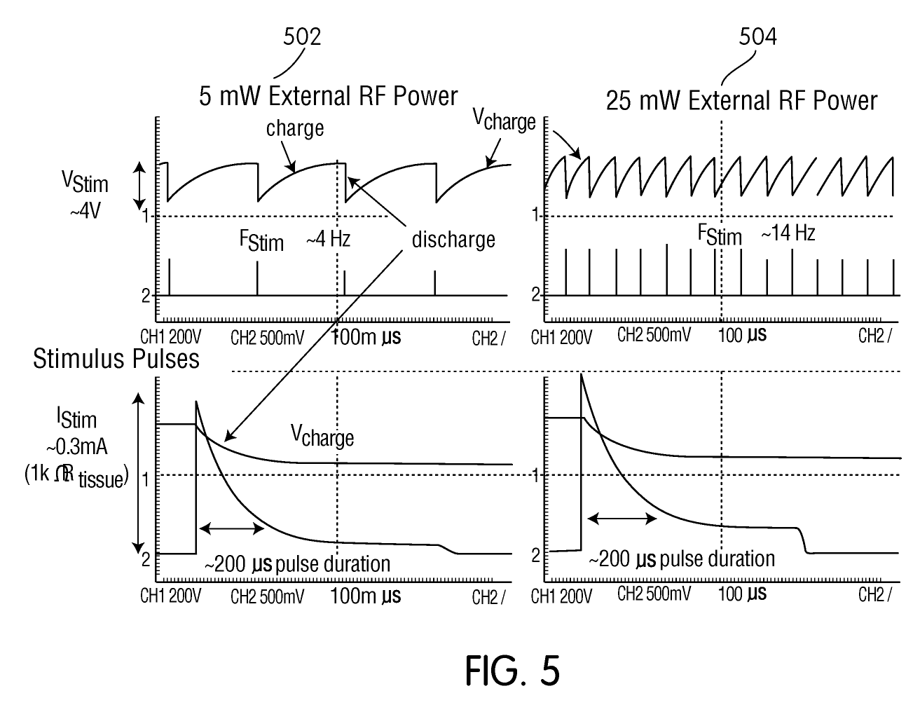

FIG. 5 presents several graphs that illustrate how wireless microtransponder stimulus frequencies, stimulus current peak amplitudes, and stimulus pulse durations vary under different device settings and external RF power input conditions, in accordance with one embodiment. In the first graph 502, the external RF power input is set at 5 mW resulting in a stimulus frequency of 4 Hz. As discussed previously, the stimulus frequency is a function of RF power as it directly affects the time it takes to charge up to the stimulus voltage. This direct relationship between RF power and stimulus frequency is clearly shown in graph 502 compared to graph 504, where the external RF power is ramped up from 5 mW to 25 mW, which results in a significant increase in stimulus frequency from 4 Hz to 14 Hz. It should be understood, however, that these are just examples of how RF power input settings affect stimulus frequency. In practice, the effects of the RF power input setting on stimulus frequency may be magnified or diminished depending on the particular application (e.g., depth of implantation, proximity to interfering body structures such as bones, organs, etc.) and device settings.

While RF intensity controls stimulus frequency, the stimulus voltage is typically controlled by the transponder zener diode element. The effect of stimulus voltage upon the stimulus current peak amplitude and pulse duration is further determined by the resistive properties of the tissue surrounding the microtransponder.

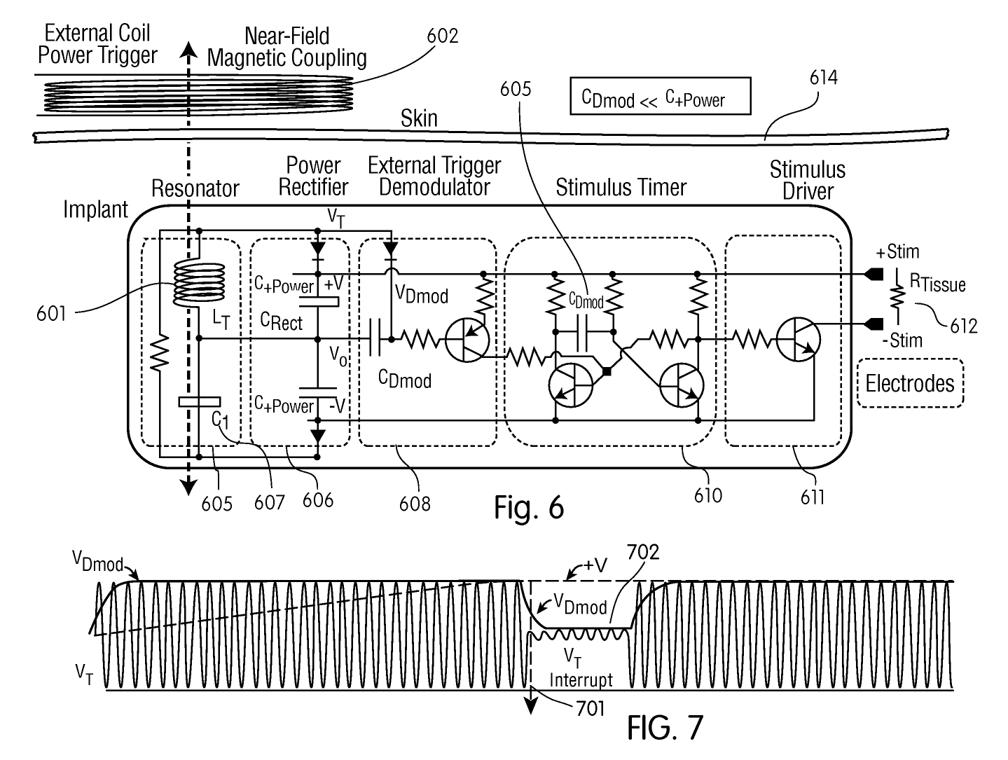

FIG. 6 is an illustration of a circuit diagram for a wireless microtransponder with an external trigger signal de-modulator element to synchronize the stimuli delivered with a plurality of other wireless microtransponders, in accordance with one embodiment. As depicted, herein, the wireless microtransponder design of FIG. 5 is modified to include an external trigger signal demodulator element 608 so that the stimulus discharge can be synchronized by a trigger signal from an external RF power field.

The modified circuit includes a resonator element 604, a rectifier element 606, an external trigger demodulator element 608, a stimulus timer element 610, a stimulus driver element 611, and one or more electrodes 612. The resonator element 604 includes a coil (L_{T}) component 601 that is coupled to a capacitor (C_{T}) component 607. The resonator element 604 is configured to oscillate at a precise frequency that depends upon the values of these two components (i.e., the coil component 601 and capacitor component 607) as described in Equation 1.

The resonator element 604 is coupled to the rectifier element 606, which is in turn coupled to the external trigger demodulator element 608, the stimulus timer element 610, and the stimulus driver element 611. The rectifier element 606 and the stimulus timer element 608 are both coupled in parallel to the capacitor 607. In addition, the stimulus driver element 611 is coupled to electrodes 612 (gold or Platinum-iridium alloy), thereby electrically connecting the stimulus driver element 611 to neural conduction tissue (axons).

It should be appreciated that in certain embodiments, a voltage booster component (not shown) can be inserted immediately after the rectifier element 606 to boost the supply voltage available for stimulation and operation of integrated electronics beyond the limits generated by the miniaturized LC resonant ‘tank’ circuit (i.e. the coil component 601 and capacitor component 607). This voltage booster can enable electro-stimulation and other microtransponder operations using the smallest possible LC components which may generate too little voltage (<0.5V). Examples of high efficiency voltage boosters include charge pumps and switching boosters using low-threshold Schottky diodes. However, it should be understood that any type of conventional high efficiency voltage booster may be utilized in this capacity as long as it can generate the voltage required by the particular application that the microtransponder is applied to.

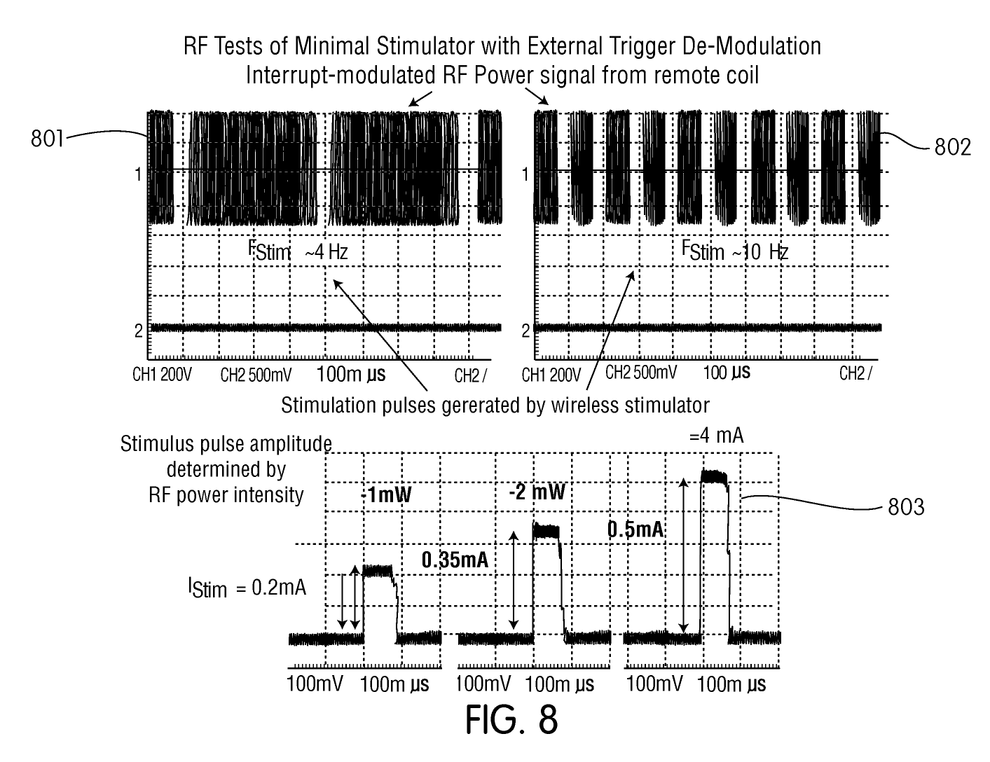

As shown in FIG. 7, the external synchronization-trigger circuit configuration (shown in FIG. 6) can employ a differential filtering method to separate the trigger signal, consisting of a sudden power interruption 701, from the slower drop in transponder power voltage 702 during the interruption. In particular, the circuit configuration (in FIG. 6) can utilize a separate capacitor (C_{Dur}) 605, in the stimulus timer element 610, to set the stimulus duration using a mono-stable multi-vibrator. Stimulus intensity can be controlled externally by the intensity of the applied RF power field generated by the external RF power coil 602. As the RF power field is modulated, the timing and frequency of stimuli from all the microtransponders under the external RF power coil 602 are synchronized externally.

Using the external synchronization-trigger circuit configuration (shown in FIG. 6), the degree of spatio-temporal control of complex stimulus patterns is essentially unlimited. In certain embodiments, the circuit configuration of the external synchronization-trigger circuit can be further modified so that it is configured to de-modulate the unique identity code of each microtransponder. This essentially permits the independent control of each microtransponder via RF signals. This added capability can provide a method to mediate the spatio-temporal dynamics necessary to restore natural sensations with artificial limbs or enable new sensory modalities (e.g., feeling infrared images, etc.).

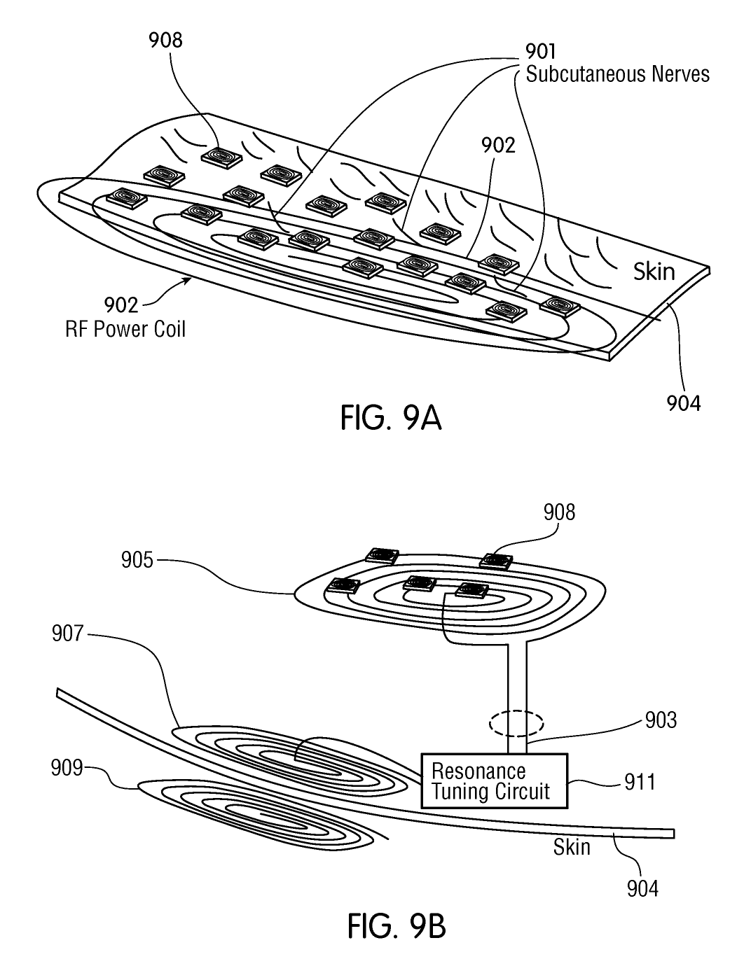

FIG. 8 presents several graphs that summarize the results from tests of a wireless microtransponder (with an external interrupt trigger de-modulator element) under different device settings and external RF power input conditions, in accordance with one embodiment. In the first graph 801, the external RF power coil modulates the RF power field to communicate a first trigger signal setting, which results in a stimulus frequency of 2 Hz. As discussed previously, the stimulus frequency is controlled by a trigger signal created when the RF power coil modulates the RF power field. The stimulus frequency is therefore directly related to the RF power field modulation frequency as shown in the second graph 802, where the stimulus frequency equals 10 Hz.

Whereas the stimulus frequency is controlled by external RF power field modulation settings, the stimulus current peak amplitude is controlled by the RF power intensity setting, as shown in the third graph 803. That is, the stimulus current peak amplitude is directly related to the RF power intensity setting. For example, an RF power intensity setting of 1 mW produces a stimulus current peak amplitude of 0.2 mA, a RF power intensity setting of 2 mW produces a stimulus current peak amplitude of 0.35 mA, and a RF power intensity setting of 4 mW produces a stimulus current peak amplitude of 0.5 mA. It should be understood, however, that these are just examples of how RF power intensity setting affects stimulus current peak amplitude. In practice, the effects of the RF power intensity setting on stimulus current peak amplitude may be magnified or diminished depending on the particular application (e.g., depth of implantation, proximity to interfering body structures such as bone, etc.) and device settings.

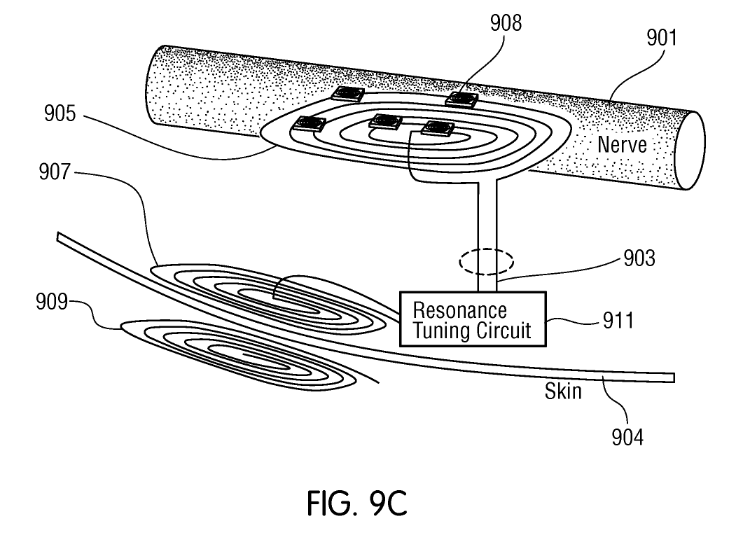

FIG. 9A is an illustration of a deployment of a plurality of wireless microtransponders distributed throughout subcutaneous vascular beds and terminal nerve fields, in accordance with one embodiment. As depicted, a plurality of independent wireless microtransponders 908 are implanted subcutaneously in a spread pattern under the skin 904 over the area that is affected. In this embodiment, each microtransponder is positioned proximate to and/or interfaced with a branch of the subcutaneous sensory nerves 901 to provide electrostimulation of those nerves. In one embodiment, only synchronous microtransponders are deployed. In another embodiment only asynchronous microtransponders are deployed. In yet another embodiment a combination of synchronous and asynchronous microtransponders are deployed.

After the deployment of the microtransponders, electrostimulation can be applied by positioning a RF power coil 902 proximate to the location where the microtransponders are implanted. The parameters for effective electrostimulation may depend upon several factors, including: the size of the nerve or nerve fiber being stimulated, the effective electrode/nerve interface contact, the conductivity of the tissue matrix, and the geometric configuration of the stimulating fields. While clinical and empirical studies have determined a general range of suitable electrical stimulation parameters for conventional electrode techniques, the parameters for micro-scale stimulation of widely distributed fields of sensory nerve fibers are likely to differ significantly with respect to both stimulus current intensities and the subjective sensory experience evoked by that stimulation.

Parameters for effective repetitive impulse stimulation using conventional electrode techniques are typically reported with amplitudes ranging to about 10 V (or up to about 1 mA) lasting up to about 1 millisecond repeated up to about 100 pulses/s for periods lasting several seconds to a few minutes at a time. In an exemplary embodiment effective repetitive impulse stimulation can be achieved with an amplitude of less than 100 μA and stimulation pulses lasting less than 100 μs.

FIG. 9B is an illustration of a deployment of wireless microtransponders to enable coupling with deep microtransponder implants, in accordance with one embodiment. As shown herein, two simple electrical wires 903 lead from the subdermal/subcutaneous implanted outer transfer coil 907 to the deeper subcutaneous implanted inner transfer coil 903 proximate to a field of implanted micro-transponders 908. Threading the wires 903 through the interstitial spaces between muscles and skin involves routine minimally invasive surgical procedures as simple as passing the lead through hypodermic tubing, similar to routine endoscopic methods involving catheters. The minimal risks of such interstitial wires 903 are widely accepted.

The deep inner transfer coil 905 is implanted to couple with the deeply implanted field of microtransponders 908 located near deep targets of micro-stimulation, such as deep peripheral nerves, muscles or organs such as the bladder or stomach as needed to treat a variety of clinical applications and biological conditions. The inner transfer coil 905 is tuned to extend the resonance of the external coil 909 to the immediate vicinity of the implanted micro-transponders 908 for maximal coupling efficiency. In addition to extending the effective range of the microtransponder 908 implants, the inner transfer coil 905 also provides another wireless link that can preserve the integrity of any further protective barrier around the target site. For instance, the inner transfer coil 905 can activate micro-transponders 908 embedded within a peripheral nerve without damaging the epineurium that protects the sensitive intraneural tissues. To ensure optimal tuning of the transfer coils (e.g., the outer transfer coil 907 and inner transfer coil 905), a variable capacitor or other tuning elements in a resonance tuning circuit 911 are added to the outer transfer coil 907 where it can be implanted with minimal risk of tissue damage. In certain embodiments, this resonance tuning circuit 911 is required, while in others it is unnecessary.

FIG. 9C is an illustration of a deployment of wireless microtransponders to enable coupling with deep neural microtransponder implants, in accordance with one embodiment. As shown herein, an extraneural inner transfer coil 905 positioned proximate to (or interfaced with) a nerve fiber or cell cluster 901 is interconnected to an outer transfer coil 907 by a simple pair of leads 903 that mediate all the signals and power necessary to operate micro-transponders 908 implanted anywhere in the body, beyond the direct effective range of powering by any external coil 909 (e.g., epidermal coil, etc.). In certain embodiments, the subdermal outer transfer coil 907 is tuned to the external coil 909 and implanted immediately under the external coil 909 just below the surface of the skin 904 for maximum near-field wireless magnetic coupling. This allows the RF waves generated by the external coil 909 to penetrate the body without long-term damage to the skin 904 and the risk of infection. In other embodiments, the outer transfer coil 907 is tuned to the external coil 909 and implanted deeper in the tissue subcutaneously. In some embodiments, a resonance tuning circuit 911 is required interposed between the inner transfer coil 905 and the outer transfer coil 907 to adjust the frequency of the signal at inner transfer coil 905, while in others it is unnecessary.

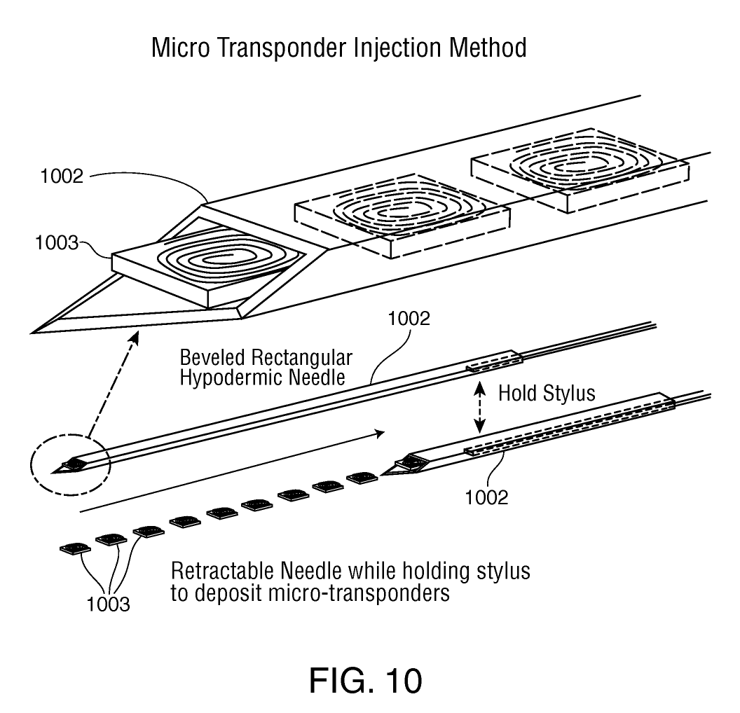

FIG. 10 is an illustration of how wireless microtransponders can be implanted using a beveled rectangular hypodermic needle, in accordance with one embodiment. As shown, the needle 1002 is curved to conform to the transverse cervical curvature (bevel concave) and without further dissection is passed transversely in the subcutaneous space across the base of the affected peripheral nerve tissue. Rapid insertion usually negates the need for even a short active general anesthetic once the surgeon becomes familiar with the technique. Following the placement of the microtransponders 1003 from the needle 1002, the needle 1002 is carefully withdrawn and the electrode placement and configuration can be evaluated using intraoperative testing. Electrostimulation can be applied using a temporary RF transmitter placed proximate to the location where the microtransponders 1003 are implanted, so the patient can report on the stimulation location, intensity, and overall sensation.

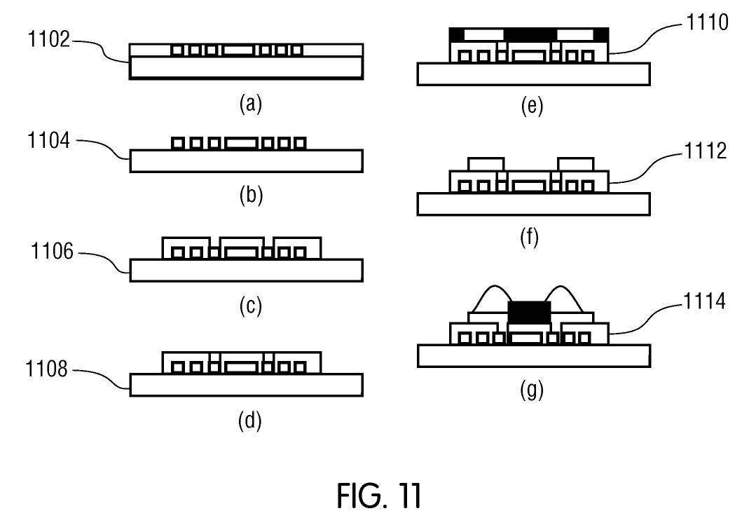

FIG. 11 is an illustration of a fabrication sequence for spiral type wireless microtransponders, in accordance with one embodiment. At step 1102, a layer of gold spiral coil is electroplated onto a substrate (typically a Pyrex® based material, but other materials may also be used as long as they are compatible with the conducting material used for the spiral coil and the particular application that the resulting microtransponder will be applied to). Electroplated gold is used as the conductor material due to its high conductivity, resistance to oxidation, and proven ability to be implanted in biological tissue for long periods of time. It should be appreciated, however, that other conducting materials can also be used as long as the material exhibits the conductivity and oxidation resistance characteristics required by the particular application that the microtransponders would be applied to. Typically, the gold spiral coil conductors have a thickness of between approximately 5 μm to approximately 25 μm.

In one embodiment, the gold spiral coil takes on a first configuration where the gold conductor is approximately 10 μm wide and there is approximately 10 μm spacing between the windings. In another embodiment, the gold spiral coil takes on a second configuration where the gold conductor is approximately 20 μm wide and there is approximately 20 μm spacing between the windings. As will be apparent to one of ordinary skill in the art, however, the scope of the present invention is not limited to just these example gold spiral coil configurations, but rather encompasses any combination of conductor widths and winding spacing that are appropriate for the particular application that the coil is applied to.

In step 1104, the first layer of photoresist and the seed layer are removed. In one embodiment, the photoresist layer is removed using a conventional liquid resist stripper to chemically alter the photoresist so that it no longer adheres to the substrate. In another embodiment, the photoresist is removed using a plasma ashing process.

In step 1106, an isolation layer of SU-8 photo resist is spun and patterned to entirely cover each spiral inductor. Typically, the SU-8 layer has a thickness of approximately 30 μm. In step 1108, a top seed layer is deposited on top of the SU-8 isolation layer using a conventional physical vapor deposition (PVD) process such as sputtering. In step 1110, a top layer of positive photoresist coating is patterned onto the top seed layer and the SU-8 isolation layer, and in step 1112, a layer of platinum is applied using a conventional electroplating process. In step 1114, a chip capacitor and a RFID chip are attached to the platinum conducting layer using epoxy and making electrical connections by wire bonding. In certain embodiments, the capacitor has a capacitance rating value of up to 10,000 picofarad (pF).

It is possible to implant such small microtransponders by simply injecting them into the subdermal tissue. Using local anesthesia at the injection site, the patient may be positioned laterally or prone depending on the incision entry point. The subdermal tissues immediately lateral to the incision are undermined sharply to accept a loop of electrode created after placement and tunneling to prevent electrode migration. A Tuohy needle is gently curved to conform to the transverse posterior cervical curvature (bevel concave) and without further dissection is passed transversely into the subdermal space across the base of the affected peripheral nerves. Rapid needle insertion usually obviates the need for even a short acting general anesthetic once the surgeon becomes facile with the technique. Following placement of the electrode into the Tuohy needle, the needle is withdrawn and the electrode placement and configuration is evaluated using intraoperative testing.

After lead placement, stimulation is applied using a temporary RF transmitter to various select electrode combinations enabling the patient to report on the table the stimulation location, intensity and overall sensation. Based on prior experience with wired transponders, most patients should report an immediate stimulation in the selected peripheral nerve distribution with voltage settings from 1 to 4 volts with midrange pulse widths and frequencies. A report of burning pain or muscle pulling should alert the surgeon the electrode is probably placed either too close to the fascia or intramuscularly.

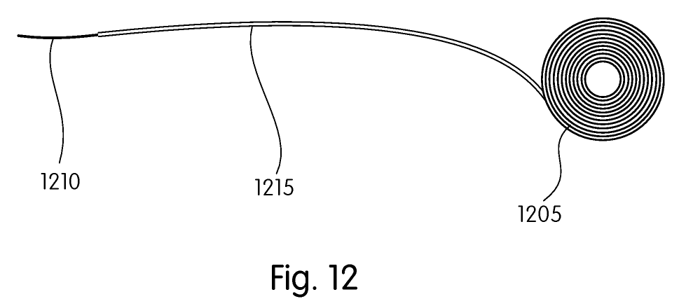

FIG. 12 is an illustration of an exemplary inner and outer transfer coil assembly using a coax cable. A first spiral coil 1205 formed from 30 gauge (0.255 mm diameter) magnetic wire is coupled to an identically formed second spiral coil 1210 (edge view). The coupling wire in this example is 15 cm of Belden 83265 coaxial cable (1.7 mm diameter, 50 ohm), which was found to be suitable for transmitting high frequency signals. The assembly of this embodiment dispenses with a tuning circuit, so the power frequency transmitted in this exemplary embodiment would be at the resonant or a critical frequency of the resonator circuit in the powered microtransponders as well as the spiral coils 1205 and 1210.

Of course, the innovations of the present application are not limited to the embodiments disclosed, but can include various materials, configurations, positions, or other modifications beyond these embodiments shown, which are exemplary only.

According to various embodiments, there is provided: a wireless transponder system for deep implantation in a patient, comprising: a first biocompatible coil; an electrical connection coupling said first biocompatible coil to a second biocompatible coil; and a biocompatible microtransponder wirelessly coupled to said second biocompatible coil; wherein said microtransponder is powered by said second biocompatible coil, using power coupled through said electrical connection from said first biocompatible coil.

According to various embodiments, there is provided: a deep implantation transponder system, comprising: an outer transfer coil implanted proximate to the skin; an inner transfer coil implanted proximate to one or more microtransponders implanted at least proximate to biological tissue, said outer transfer coil and said inner transfer coil being electrically coupled together; and the outer transfer coil tuned to an external power coil for near field magnetic coupling allowing power from the external coil to power said microtransponders.

According to various embodiments, there is provided: a system for a wireless deep implantation of one or more transponders in a patient, comprising: one or more wireless microtransponders; a first coil positioned subdermally and electrically coupled to a second coil proximate to the microtransponders, wherein the second coil can inductively couple with ones of said microtransponders; and said microtransponders are wirelessly coupled to and powered by said second coil.

According to various embodiments, there is provided: a method for operating a wireless deep implantation unit in a patient, comprising the steps of: distributing one or more electronic units within a desired volume internally; positioning a first coil proximate to a surface of the body, and coupled to a second coil proximate to the one or more electronic units; and powering said electronic units using a wireless connection to the second coil resonating at a frequency which is harmonically related to a resonant frequency of a resonator power circuit in ones of said electronic units.

According to various embodiments, there is provided: a method for using a deep implantation transponder in a patient, comprising the steps of: positioning a first coil proximate to the surface of the body and coupled to a second coil proximate to a plurality of microtransponders distributed within a deep tissue area; and powering said microtransponders using the second coil with a power signal waveform which includes at least one harmonically related frequency of a resonator power circuit in at least ones of said microtransponders.

According to various embodiments, there is provided: a method for powering a deep implantation transponder in a patient, comprising the steps of: coupling a subdermal outer transfer coil to an inner transfer coil located proximate to a plurality of microtransponders; and driving the inner transfer coil at a resonant or harmonic frequency of a resonator power circuit in said microtransponders to power said microtransponders.

According to various embodiments, there is provided: a deep transponder system, comprising: a plurality of outer transfer coils implanted underneath the skin coupled to at least one of a plurality of inner transfer coils implanted proximate to a plurality of microtransponders implanted in tissue; individual ones of the transfer coils tuned to at least one of a plurality of external power coils for near field magnetic coupling allowing radio frequency power from the external coils to power selected microtransponders at predetermined resonant or harmonic frequencies.

According to various embodiments, there is provided: a deep transponder system, comprising: a plurality of outer transfer coils coupled electrically with a plurality of inner transfer coils positioned proximate to a plurality of microtransponders implanted in tissue; and respective ones of the outer transfer coils being inductively coupled to a movable external power coil to thereby allow radio frequency power from the external coils to power selected microtransponders at predetermined tuned frequencies.

According to various embodiments, there is provided: a method for operating a deep implantation transponder, comprising the steps of: implanting an outer transfer coil in subdermal tissue coupled to an inner transfer coil implanted proximate to a plurality of microtransponders in tissue; and coupling the outer transfer coil to an epidermal power coil using wireless near field magnetic coupling to thereby allow radio frequency power from the epidermal power coil to power said microtransponders.

According to various embodiments, there is provided: a method for operating a deep nerve transponder, comprising the steps of: coupling a plurality of microtransponders interfaced with a plurality of deep nerves to a proximately implanted first coil using wireless magnetic coupling, said first coil connected to a second coil implanted in subdermal tissue; and powering the microtransponders by coupling the second coil to an epidermal third coil using wireless near field magnetic coupling and transmitting radio frequency power from the epidermal third coil.

According to various embodiments, there is provided: a tissue-implantable transfer unit for implanted wireless microtransponders, comprising: first and second biocompatible coils; and a biocompatible electrical connection coupling said first and second coil; wherein said first and second coils form a coupled passive resonator; and whereby said first and second coils jointly provide power transfer from wireless power inputs at said first coil to wireless power outputs at said second coil.

According to various embodiments, there is provided: a method for powering a wireless transponder system in a patient, comprising the steps of: providing a first biocompatible coil; establishing an electrical connection coupling the first biocompatible coil to a second biocompatible coil; and coupling a biocompatible microtransponder wirelessly to the second biocompatible coil; wherein the microtransponder is powered by the second biocompatible coil using power coupled through the electrical connection from the first biocompatible coil.

### MODIFICATIONS AND VARIATIONS

As will be recognized by those skilled in the art, the innovative concepts described in the present application can be modified and varied over a tremendous range of applications, and accordingly the scope of patented subject matter is not limited by any of the specific exemplary teachings given.

The specific implementations given herein are not intended to limit the practice of the present innovations.

One such specific variation is dispensing with the subdermal/outer transfer coil to use a three coil power transmission arrangement. Power from the external coil would transmit to the subcutaneous/inner transfer coil to power the microtransponder micro-coil.

The interface between the two transfer coils can comprise radio frequency, low frequency, or direct current power. The wired connection between the two transfer coils can typically be coaxial or a balanced line connection. The external coil and the subdermal/outer transfer coil can comprise paralleled coils at the skin surface. There can further be multiple internal drivers to power the microtransponders. The configuration can also make use of spatial resolution. Finally, the described embodiment is a single power transfer through one internal tissue boundary, while the invention also extends to a double power transfer through two internal boundaries or potentially more.

It is also possible to vary the power source in the invention as mentioned above, so the power is not limited to RF power. The connection between the subdermal (or outer transfer) coil and subcutaneous (or inner transfer) coil does not necessarily have to be a connection using power transfer at the resonant RF frequency. In alternative embodiments, it is contemplated that this power-transfer connection can be DC, or can be AC at a lower frequency than RF, or at a non-resonating AC frequency of the microtransponder micro-coils.

If the connection is DC, a power conversion stage would be included in the outer transfer coil circuitry or on the wire link to the inner transfer coil to convert the received RF power to DC. This can be quite similar to an AC-DC conversion used to charge up a storage capacitor for stimulation pulses. In this example, the inner transfer coil would need to contain, or be combined with, an oscillator circuit to generate an AC signal (for wireless coupling) from the received DC power. The AC signal then permits wireless coupling of power from the inner transfer coil to the microtransponder power circuits.

Similar adaptation can be used with the connecting link operating at a lower AC frequency or non-resonating AC frequency. In either of these embodiments, a conversion stage circuit would be included in the inner transfer coil circuitry or on the wire link to the inner transfer coil to convert the received low frequency or non-resonate AC power signal into an AC signal compatible with powering the microtransponders power circuits (e.g. a resonant or other critical frequency for the resonator circuit).

The following applications may contain additional information and alternative modifications: Attorney Docket No. MTSP-29P, Ser. No. 61/088,099 filed Aug. 12, 2008 and entitled “In Vivo Tests of Switched-Capacitor Neural Stimulation for Use in Minimally-Invasive Wireless Implants; Attorney Docket No. MTSP-30P, Ser. No. 61/088,774 filed Aug. 15, 2008 and entitled “Micro-Coils to Remotely Power Minimally Invasive Microtransponders in Deep Subcutaneous Applications”; Attorney Docket No. MTSP-31P, Ser. No. 61/079,905 filed Jul. 8, 2008 and entitled “Microtransponders with Identified Reply for Subcutaneous Applications”; Attorney Docket No. MTSP-33P, Ser. No. 61/089,179 filed and entitled “Addressable Micro-Transponders for Subcutaneous Applications”; Attorney Docket No. MTSP-36P Ser. No. 61/079,004 filed Jul. 8, 2008 and entitled “Microtransponder Array with Biocompatible Scaffold”; Attorney Docket No. MTSP-38P Ser. No. 61/083,290 filed Jul. 24, 2008 and entitled “Minimally Invasive Microtransponders for Subcutaneous Applications” Attorney Docket No. MTSP-39P Ser. No. 61/086,116 filed Aug. 4, 2008 and entitled “Tintinnitus Treatment Methods and Apparatus”; Attorney Docket No. MTSP-40P, Ser. No. 61/086,309 filed Aug. 5, 2008 and entitled “Wireless Neurostimulators for Refractory Chronic Pain”; Attorney Docket No. MTSP-41P, Ser. No. 61/086,314 filed Aug. 5, 2008 and entitled “Use of Wireless Microstimulators for Orofacial Pain”; Attorney Docket No. MTSP-42P, Ser. No. 61/090,408 filed Aug. 20, 2008 and entitled “Update: In Vivo Tests of Switched-Capacitor Neural Stimulation for Use in Minimally-Invasive Wireless Implants”; Attorney Docket No. MTSP-43P, Ser. No. 61/091,908 filed Aug. 26, 2008 and entitled “Update: Minimally Invasive Microtransponders for Subcutaneous Applications”; Attorney Docket No. MTSP-44P, Ser. No. 61/094,086 filed Sep. 4, 2008 and entitled “Microtransponder MicroStim System and Method”; Attorney Docket No. MTSP-28, Ser. No. ______, filed ______ and entitled “Implantable Transponder Systems and Methods”; Attorney Docket No. MTSP-31, Ser. No. ______, filed and entitled “Implantable Driver with Charge Balancing”; Attorney Docket No. MTSP-32, Ser. No. ______, filed ______ and entitled “A Biodelivery System for Microtransponder Array”; Attorney Docket No. MTSP-46, Ser. No. ______, filed ______ and entitled “Implanted Driver with Resistive Charge Balancing”; Attorney Docket No. MTSP-47, Ser. No. ______, filed ______ and entitled “Array of Joined Microtransponders for Implantation”; and Attorney Docket No. MTSP-48, Ser. No. ______, filed ______ and entitled “Implantable Transponder Pulse Stimulation Systems and Methods” and all of which are incorporated by reference herein.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

The claims as filed are intended to be as comprehensive as possible, and NO subject matter is intentionally relinquished, dedicated, or abandoned.

1. A wireless transponder system for deep implantation in a patient, comprising:
a first biocompatible coil;
an electrical connection coupling said first biocompatible coil to a second biocompatible coil; and
a biocompatible microtransponder wirelessly coupled to said second biocompatible coil;
wherein said microtransponder is powered by said second biocompatible coil, using power coupled through said electrical connection from said first biocompatible coil.

2. The system of claim 1, wherein said first biocompatible coil is under a skin surface, and closer thereto than said second biocompatible coil is.

3. The system of claim 1, further comprising:
the first biocompatible coil receiving radio frequency power matching a critical frequency of a micro-coil and a resonator circuit within at least one microtransponder.

4. The system of claim 1, further comprising:
an external coil transmitting a radio frequency power signal to said first biocompatible coil.

5. The system of claim 1, further comprising:
an external coil transmitting a radio frequency signal at a resonant or harmonic frequency of a resonator power circuit in at least one microtransponder to the first biocompatible coil.

6. The system of claim 1, further comprising:
the first biocompatible coil receiving a radio frequency power signal to transmit to the second biocompatible coil to wirelessly power at least one microtransponder.

7. The system of claim 6, further comprising:
a resonance tuning circuit positioned between the first and second biocompatible coil.

8. The system of claim 1, further comprising:
the power signal received by the first biocompatible coil converted to a direct current signal; and
a converter circuit transforming the direct current signal to an alternating current signal compatible for power transfer from the second biocompatible coil to wirelessly power at least one transponder.

9. A deep implantation transponder system, comprising:
an outer transfer coil implanted proximate to the skin;
an inner transfer coil implanted proximate to one or more microtransponders implanted at least proximate to biological tissue, said outer transfer coil and said inner transfer coil being electrically coupled together; and
the outer transfer coil tuned to an external power coil for near field magnetic coupling allowing power from the external coil to power said microtransponders.

10. The system of claim 9, wherein the inner transfer coil receives radio frequency power at a critical frequency of a micro-coil and a resonator circuit within at least one microtransponder.

11. The system of claim 9, wherein the external power coil transmits a radio frequency signal tuned to match a resonant or harmonic frequency of a resonator power circuit in at least one microtransponder.

12. The system of claim 9, wherein the outer transfer coil receives a radio frequency signal at a select frequency to generate an alternating current signal tuned to power at least one microtransponder.

13. The system of claim 9, further comprising:
a resonance tuning circuit interposed between the outer transfer coil and the inner transfer coil.

14. The system of claim 9, wherein the inner transfer coil receives power at a resonant or harmonic frequency of a resonator power circuit within a microtransponder comprised of at least a micro-coil and a resonator circuit.

15-31. (canceled)

32. A method for powering a deep implantation transponder in a patient, comprising the steps of:
coupling a subdermal outer transfer coil to an inner transfer coil located proximate to a plurality of microtransponders; and
driving the inner transfer coil at a resonant or harmonic frequency of a resonator power circuit in said microtransponders to power said microtransponders.

33. The method of claim 32, wherein the subdermal outer transfer coil receives a power signal from an exterior coil.

34. The method of claim 33, wherein the power signal is an alternating current signal transmitted at the resonant or harmonic frequency.

35. The method of claim 33, wherein the power signal is converted to the resonant or harmonic frequency using a converter circuit interposed between the outer transfer coil and the inner transfer coil.

36. The method of claim 33, wherein the power signal is transmitted at the resonant or harmonic frequency of the inner transfer coil to induce an alternating current from the inner transfer coil.

37. The method of claim 32, wherein an inner transfer coil alternating current power signal is tuned to the resonant or harmonic frequency.

38-59. (canceled)
